(19) United States
(12) Patent Application Publication
(10) Pub. No.: US 2017/0172880 A1
(43) Pub. Date: Jun. 22, 2017

(54) Fast Dissolving Peroxymonosulfate Composition

(71) Applicant: Colgate-Palmolive Company, New York, NY (US)

(72) Inventors: Stacey Lavender, Chesterfield, NJ (US); Guofeng Xu, Plainsboro, NJ (US); Cajetan Dogo-Isonagie, Highland Park, NJ (US)

(73) Assignee: Colgate-Palmolive Company, New York, NY (US)

(21) Appl. No.: 15/375,320

(22) Filed: Dec. 12, 2016

Related U.S. Application Data
(60) Provisional application No. 62/269,488, filed on Dec. 18, 2015.

(51) Int. Cl. A61K 8/46; A61K 8/02; A61Q 11/00; A61K 8/81; A61K 8/365; A61K 8/36; A61K 8/86

(52) U.S. Cl. CPC A61K 8/463; A61K 8/36; A61K 8/0204; A61K 8/86; A61K 8/8176; A61K 8/365; A61Q 11/00; A61K 2800/882; A61K 2800/594

(57) ABSTRACT

A solid composition for adding to water of an aqueous solution to form a whitening mouthrinse. The solid composition can be in the form of a tablet and contains a salt of peroxymonosulfate as a whitening agent.

### CROSS-REFERENCE T CROSS-REFERENCE TO RELATED PATENT APPLICATIONS

This application claims the benefit of priority to U.S. Provisional Patent Application Ser. No. 62/269,488 filed Dec. 18, 2015, the entirety of which is incorporated herein by reference.

### RELATED PATENT APPLICATIONS

This application claims the benefit of priority to U.S. patent application Ser. No. 14/797,327, filed Jul. 13, 2015 and to U.S. Provisional Patent Application Ser. No. 62/089,918 filed Dec. 10, 2014, the entireties of which are incorporated herein by reference.

### BACKGROUND

Potassium peroxymonosulfate is a powerful oxidizing and stain removing agent and is currently used in denture cleaning tablets. Typical denture cleaning tablets are quite large weighing about 2.6 g. They are intended to be dissolved in a cup of water (about 200 ml). The dissolve time is on the order of minutes and would be longer in smaller volumes. After dissolving the tablet there still can be some particulates that remain.

Mouthrinses or mouthwashes having tooth whitening effects are desirable. A typical volume of water or aqueous solution for mouthrinse applications is about 15 ml. It would be desirable to have a tablet containing a whitening agent in a solid, e.g., tablet form, which is fast dissolving in water that can be easily used by consumers in mouthrinse applications.

### BRIEF SUMMARY

The invention concerns a solid composition, e.g., a tablet or powder, containing an oxidizing agent comprising inorganic salts of peroxymonosulfate, preferably alkaline metal salts and alkaline earth metal salts or mixtures thereof. The solid composition is quick-dissolving that can be added to water or an aqueous solution to be then used by a consumer as a whitening mouthrinse. Once dissolved the consumer would use the resulting liquid as a typical mouthrinse.

Thus, the present invention concerns a solid composition comprising:
(a) a tooth whitening effective amount of an inorganic salt of peroxymonosulfate, in particular, potassium peroxymonosulfate,
(b) a buffering agent, and optionally
(c) a disintegrating agent;
wherein the solid composition completely dissolves when placed in water.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Open terms such as “include,” “including,” “contain,” “containing” and the like mean “comprising.” In this description, unless otherwise stated, the use of the singular also includes the plural. For example, “a lubricant” also comprehends the case where more than one lubricant is used

The solid composition of the invention comprises an inorganic salt, such as a metal salt, of peroxymonosulfate. Such salts are preferably alkaline metal salts and alkaline earth metal salts or mixtures thereof. Specific examples of such salts are sodium peroxymonosulfate, potassium peroxymonosulfate, ammonium peroxymonosulfate, and the like.

One embodiment of the peroxymonosulfate is potassium peroxymonosulfate (also known as MPS, potassium monopersulfate). The potassium peroxymonosulfate (an example of which is Oxone®, an oxidizing agent) may be combined to form or exist as a trisalt of potassium peroxymonosulfate, potassium hydrogen sulfate and potassium sulfate (2KHSO₅.KHSO₄.K₂SO₄).

Potassium peroxymonosulfate has limited stability in aqueous solutions and in other common toothpaste ingredients. Therefore contact with water during processing and storage should be avoided or minimized. The solid form of the invention overcomes the stability concerns of the oxidizing agent as long as the tablet is packaged in a moisture free environment. The solid composition, e.g., tablet, granules or powder, is individually packaged and sealed in each unit dose.

The solid composition is typically stored in an air tight, moisture-proof package including, e.g., sealed metal foil pouches, blister packs, and desiccant capped tubes. Useful packaging materials include, e.g., polymeric packaging (e.g., polyethylene and polypropylene), metal foils (e.g., aluminum), and combinations thereof.

The composition of the invention completely dissolves in 15 ml of water at 23° C. in 1 minute or less with minimal to moderate agitation. The phrase “completely dissolves” means that the resulting solution has no visible particulate material, sediment or solid. The phrase “minimal to moderate agitation” means shaking or swirling of the mixture by hand by the consumer in a suitable container such as a cup or glass. In some embodiments the solid composition dissolves in 50 seconds or less, 40 seconds or less, 30 seconds or less, or 20 seconds or less when added to 15 ml or water or an aqueous solution, e.g., a mouthrinse base.

The solid compositions of the invention contain no water or have a low water content. As used herein, the term “low water content” means the total concentration of water, including any free water and all water contained in any ingredients. In various embodiments of the composition, the amount of water is in an amount of less than 4% by weight, or less than 3% by weight, or less than 2% by weight, or less than 1% by weight, or less than 0.5% by weight, or about 0.001% to about 4% by weight, or about 0.0001% to about 0.5% by weight or about 0.0001% to about 0.1% by weight.

The solid composition of the invention can be in a variety of forms including, e.g., powder (e.g., a free flowing granulation), tablet, caplet (type of tablet), granule, pellet, wafer, film and bead. Typical tablets have an initial hardness (i.e., a hardness immediately after manufacture) of at least 3 kilopounds (Kp), at least 4 Kp, from about 5 Kp to about 15 Kp, or even from about 5 Kp to about 10 Kp, as measured on a standard hardness tester fitted with a strain gauge, and a hardness 24 hours after manufacture of at least 5 Kp, at least 6 Kp, at least 10 Kp, at least 15 Kp, at least 20 Kp, or even from about 30 Kp to about 45 Kp.

The compositions of the invention, e.g., tablets, can be formed to have any desired weight and dimension. Typical composition, e.g., tablet, weights include from 0.05 gram to 5 gram, from 100 milligrams (mg) to 1000 mg, from 100 mg to 500 mg, or even from 200 mg to 400 mg. Useful tablets are also formed with a diameter of at least 5 millimeters (mm), from 5 mm to about 50 mm, from 5 mm to about 30 mm or even from about 5 mm to about 20 mm, and a thickness of at least about 1 mm, from about 1 to about 10 mm or about 1 to about 5 mm. In some embodiments the surface area of the compositions, e.g., tablets or beads, can be, for example, about 0.55 to about 9.5 square centimeters (cm²), or about 0.9 cm² to about 5 cm².

In certain embodiments, e.g., tablets, the compositions of the invention are layered, e.g., one layer comprising the peroxymonosulfate salt and one or more additional layers comprising other ingredients. In some embodiments the solid compositions, e.g., tablets, are coated with a suitable coating.

The amount of peroxymonosulfate salt, e.g., potassium peroxymonosulfate, in the solid compositions of the invention is effective to result in improved tooth whitening when used twice daily in a mouthrinse for about three months as compared to a control mouthrinse without the peroxymonosulfate salt. The amount of peroxymonosulfate salt typically is about 0.5% to about 50%, in one embodiment about 0.75% to about 40%, in another embodiment about 0.75% to about 37%, in another embodiment about 7.5% to about 15%, by weight of the total composition.

The compositions of the invention contain a buffering agent. Examples of buffering agents include anhydrous carbonates such as sodium carbonate, sesquicarbonates, bicarbonates such as sodium bicarbonate, silicates, bisulfates, phosphates such as monopotassium phosphate and dipotassium phosphate, citrates, pyrophosphates (sodium and potassium salts) and combinations thereof. The amount of buffering agent is sufficient to be effective to achieve a pH of about 5 to about 9, preferable about 6 to about 8, and more preferable about 7, when the composition is dissolved in water or a mouthrinse base. In some embodiments the amount of buffering agent is effective to achieve a pH of about 5 to about 10. Typical amounts of buffering agent are about 5% to about 35%, in one embodiment about 10% to about 30%, in another embodiment about 15% to about 25%, by weight of the total composition.

The solid compositions of the invention optionally contain a disintegrating agent, for example, when the composition is a tablet. Disintegrating agents include natural starches, such as maize starch, potato starch etc., directly compressible starches such as starch 1500, modified starches such as carboxymethyl starches and sodium starch glycolate which are available as PRIMOJEL® and EXPLOTAB® and EXPLOSOL® and starch derivatives such as amylose. Other examples are cross-linked polyvinylpyrrolidones, e.g. crospovidones available as e.g. POLYPLASDONE XL® and KOLLIDON XL®; modified celluloses such as cross-linked sodium carboxymethylcelluloses available as, e.g., AC-DI-SOL®, PRIMELLOSE®, PHARMACEL XL®, EXPLOCEL®, and NYMCEL ZSX®; alginic acid and sodium alginate; microcrystalline cellulose, e.g. AVICEL®, PHARMACEL®, EMCOCELL®, VIVAPUR®; and methacrylic acid-divinylbenzene copolymer salts available as e.g., AMBERLITE® IRP-88. Other examples of the disintegrating agent are light silicic anhydride, calcium silicate, magnesium metasilicate aluminate, and carboxymethyl cellulose. In the present invention, each of them may be used solely or two or more thereof may be used jointly. Typical amounts of disintegrating agent are about 0.5% to about 20%, in one embodiment about 1% to about 5%, in another embodiment about 1% to about 3%, by weight of the total composition.

The compositions of the invention optionally contain a binder, e.g., when the composition is a tablet, preferably a polymeric binder, that is compatible with an oxidizing agent, which adds bulk to the compositions and assists in holding the components of the composition together when in the form of a tablet. Examples of suitable polymeric binders include, e.g., starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, polyvinylpyrrolidone, pectins, alginates, polyacrylamides, polyvinyloxoazolidone, polyvinyl alcohols and mixtures thereof.

The binder can also comprise one or more non-polymeric binders such as dextrose, lactose, sucrose, sorbitol, mannitol, xylitol and the like.

Typically, the binder is present in the composition in an amount of from 10% by weight to about 60% by weight, from about 15% by weight to about 50% by weight, or even from about 25% by weight to about 40% by weight.

The solid composition of the invention is optionally an effervescent composition. The term “effervescent composition” as used herein means a composition that evolves gas bubbles when contacted with water. When the solid composition of the invention is an effervescent composition, it comprises and effervescent agent. The effervescent agent preferably is an effervescent couple that includes an acid and a base. The effervescent couple is activated when contacted with water, e.g., when the composition, e.g., tablet, is placed in a glass of water. The water liberates the acid and base and enables the acid and base to react with each other to produce carbon dioxide gas, which imparts carbonation to the aqueous composition. Examples of useful acids include citric acid, ascorbic acid, malic acid, adipic acid, tartaric acid, fumaric, succinic acid, sodium acid pyrophosphate, lactic acid, hexamic acid, and acid salts and acid anhydrides thereof, and mixtures thereof. Examples of useful acid anhydrides include citraconic anhydride, glucono-D-lactone, and succinic anhydride. Examples of useful acid salts include potassium bitartrate, acid citrate salts, sodium dihydrogen phosphate, disodium dihydrogen phosphate, sodium acid sulfite, and combinations thereof. When effervescent, the acid is present in the composition in an amount of from 10% by weight to about 60% by weight, from about 15% by weight to about 50% by weight, or even from about 25% by weight to about 40% by weight.

The base preferably is capable of generating carbon dioxide. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide and mixtures thereof. When effervescent, the base is present in the composition in an amount of from 10% by weight to about 60% by weight, from about 15% by weight to about 50% by weight, or even from about 25% by weight to about 40% by weight.

The compositions of the invention optionally contain a lubricant, e.g., when the composition is a tablet or powder. Various lubricants are suitable for use in the composition including water dispersible, water soluble, water insoluble lubricants and combinations thereof. Examples of useful water soluble lubricants include sodium benzoate, polyethylene glycol, L-leucine, adipic acid, and combinations thereof. The composition can also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water insoluble lubricants include, e.g., animal fats, polyoxyethylene monostearate, talc, and combinations thereof. When the composition is in the form of a tablet, the composition preferably includes a sufficient amount of lubricant to enable the composition to be formed into tablets and released from a high speed tableting press in the form of a tablet. Typically the amount of lubricant in the composition is from 1% by weight to about 15% by weight, from about 1% by weight to about 12% by weight, from about 2% by weight to about 10% by weight, or even from about 3% by weight to about 8% by weight. In one embodiment the composition includes sodium benzoate in an amount of from 1% by weight to about 3% by weight and polyethylene glycol in an amount of from 1% by weight to about 5.5% by weight.

The solid composition of the invention can optionally contain whitening agents in addition to the peroxymonosulfate salt. Whitening agents, material which is effective to effect whitening of a tooth surface to which it is applied, such as hydrogen peroxide and urea peroxide. In various embodiments, the compositions of this invention may optionally comprise a peroxide whitening agent, comprising a peroxide compound. A peroxide compound is an oxidizing compound comprising a bivalent oxygen-oxygen group. Peroxide compounds include peroxides and hydroperoxides, such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof. In some embodiments, the peroxide compound comprises hydrogen peroxide. In some embodiments, the peroxide compound consists essentially of hydrogen peroxide. In some embodiments a non-peroxide whitening agent may be provided. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite. One or more additional whitening agents are optionally present in a tooth-whitening effective total amount. In some embodiments the compositions additionally comprise an activator, e.g., tetraacetylethylenediamine.

The solid composition optionally can also include other ingredients including, e.g., flavor agents; fillers; surfactants; preservatives, e.g., sodium benzoate and potassium sorbate; color agents including, e.g., dyes and pigments; and sweeteners.

Examples of the surfactant that can be used are sodium lauryl sulfate, sorbitan fatty acid ester, polyoxyethylene (20) sorbitan monooleate (Polysorbate 80 or Tween 80), polyethylene glycol fatty acid ester, polyoxyethylene sorbitan fatty acid ester, polyoxyethylene alkyl ether, polyoxyethylene polyoxypropylene alkyl ether, polyoxyethylene polyoxypropylene block copolymer, polyoxyethylene alkyl phenyl ether, polyoxyethylene castor oil, polyoxyethylene hydrogenated castor oil, polyoxyethylene sorbitol fatty acid ester and polyoxyethylene glycerol fatty acid ester. In the present invention, each of them may be used solely or two or more thereof may be used jointly. Typical amounts of surfactant are about 0.5% to about 3%, in one embodiment about 0.75% to about 2%, in another embodiment about 1% to about 1.5%, by weight of the total composition.

Examples of the filler are crystalline cellulose, ethylcellulose, dextrin, various kinds of cyclodextrin (α-cyclodextrin, β-cyclodextrin and γ-cyclodextrin), sodium sulfate, as well as derivatives thereof and pullulan.

Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Useful coloring agents include, e.g., food, drug and cosmetic (FD&C) colors including, e.g., dyes, lakes, and certain natural and derived colorants. Useful lakes include dyes absorbed on aluminum hydroxide and other suitable carriers.

Useful sweetening agents include stevia, sugars such as sucrose, glucose, invert sugar, fructose, ribose, tagalose, sucralose, maltitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

It is understood that while general attributes of each of the above categories of materials may differ, there may be some common attributes and any given material may serve multiple purposes within two or more of such categories of materials. All of the ingredients in the compositions may have functions in addition to their primary function, and may contribute to the overall properties of the composition, including its stability, efficacy, consistency, mouthfeel, taste, odor and so forth. For example, a binder may also function as a disintegrating agent and vice versa.

The solid compositions of the invention can be made via techniques known in the art. Documents which disclose techniques which may be used to prepare the solid compositions of the invention are U.S. Pat. Nos. 4,886,669; 6,106,861; 6,596,311; 6,743,443; 6,811,793; 7,501,409; 7,815,897; 8,377,995; and US patent application 2005/0169986, all of which are incorporated herein by reference in their entireties. In general, the ingredients and optional components can be kneaded with an organic solvent, filled in a mold and subjected to a compression-molding. The organic solvent can be an alcohol such as methanol, ethanol, propanol, isopropanol, and the like. The kneading and granulating operations carried out by adding such auxiliary agents for making the preparation and by adding such a solvent may be conducted using the conventionally used apparatus. For example, a fluidized bed granulator, a tumbling granulator, an extrusion granulator or a spray-drying drier may be used. The solid compositions may also be prepared via freeze drying.

Powders can be prepared by compounding the ingredients and optionally calcium carbonate, and, if necessary, further orally acceptable additive(s), and mixing in a conventional manner.

Granules can be prepared by any one of known methods for preparing granules such as dry granulation, layering granulation, impregnated-granulation, etc.

For dry granulation, a mixture of ingredients with optional additive(s) is subjected to granulation with a roller compactor, a roll granulator, etc.

For layering granulation, a mixture similar to the above is added to a rolling inactive carriers while spraying a binder solution with a centrifugal fluidized bed granulator or the like to make the mixture adhere to the carries. Examples of the inactive carrier that used in this method include crystals of sugars or inorganic salts such as crystalline lactose, crystalline cellulose, crystalline sodium chloride, etc., and spherical granules such as spherical granules of crystalline cellulose (brand name: Avicel SP, Asahi Kasei Corporation), spherical granules of crystalline cellulose and lactose (brand name: Nonpareil-NP-5 and NP-7, Freund Co., Ltd.), spherical granules of purified white sugar (brand name: Nonpareil-103, Freund Co., Ltd.), spherical granules of lactose and a starch, etc.

For impregnating granulation a solution containing potassium peroxymonosulfate and other ingredients at an appropriate concentration is mixed with porous carriers thereby a sufficient amount of solution is made to retain in the cavities of the carrier, which is followed by drying to remove the solvent. Examples of the porous carrier that can be used include magnesium aluminometasilicate (bland name: Neusiline, Fuji Chemical Industry Co., Ltd.), calcium silicate (Florite, Eisai Co., Ltd.), etc. Examples of the solvent include ethanol, methanol, or the like.

Tablets can be manufactured by either subjecting a mixture prepared in the same manner as above to the compression molding as it is, or subjecting said mixture to the granulation as mentioned above, and then to the compression molding after adding disintegrant(s), lubricant(s), etc., if needed. If a carbonate is compounded, it is preferably added at the same time when a disintegrant, a lubricant, etc. are added. If desired, an additional substance(s) can be compounded.

The compression molding can be conducted using a conventional tableting machine such as rotary tableting machine, single punch tableting machine, dual tableting machine, and the like, with a compressing pressure of generally about 50 to 4,000 kg/cm2.

The present invention also concerns a method for whitening teeth comprising mixing the solid composition of the invention into water or a mouthrinse base until the composition dissolves, followed by rinsing an oral cavity containing teeth with the mouthrinse.

The term “mouthwash” or “mouthrinse” generally denotes liquid formulations which are used to rinse the surfaces of the oral cavity and provide the user with a sensation of oral cleanliness and refreshment. The mouthrinse is an oral composition that is not intentionally swallowed for purposes of systemic administration of therapeutic agents, but is applied to the oral cavity, used to treat the oral cavity and then expectorated. A mouthrinse composition will usually contain an aqueous continuous phase.

A typical mouthrinse composition consists of a liquid carrier such as water, a humectant, such as glycerin, sorbitol, propylene glycol a surfactant, such as a Pluronics, sodium lauryl sulfate, a sweetening agent, such as sodium saccharin, xylitol a flavoring agent, a coloring agent, and a preservative agent, such as potassium sorbate, sodium benzoate. The composition may also include buffering agents that have the capability to buffer to a final pH of 6.5-8, such as sodium phosphates, an anti-cavity agent, such as sodium fluoride, and an anti-bacterial agent such as cetylpyridinium chloride.

Provided is a solid composition (Composition 1) comprising (a) a tooth whitening effective amount of an inorganic salt of peroxymonosulfate, (b) a buffering agent, and optionally (c) a disintegrating agent, wherein the solid composition completely dissolves in water, e.g.,
1.1. Composition 1 wherein the composition dissolves in 1 minute or less, 50 seconds or less, 40 seconds or less, 30 seconds or less, 20 seconds or less when added to 15 ml or water or an aqueous solution, e.g., a mouthrinse base;
1.2. Composition 1 or 1.1 wherein the composition contains no water or water in an amount of less than 4%, or less than 3%, or less than 2%, or less than 1%, or less than 0.5%, or about 0001% to about 4%, or about 0.0001% to about 0.5% or about 0.0001% to about 0.1% or about 0.001% to 4%, by weight;
1.3. Any of the preceding compositions wherein the composition is in the form of a tablet, powder or granule and is packaged in a moisture free environment (e.g. in a sachet);
1.4. Any of the preceding compositions in the form of a tablet having a weight from about 0.05 gram to about 5 gram, or from about 100 milligrams (mg) to about 1000 mg, or from about 100 mg to about 500 mg, or from about 200 mg to about 400 mg, a diameter of at least 5 millimeters (mm), or from about 5 mm to about 50 mm, or from about 5 mm to about 30 mm or from about 5 mm to about 20 mm, and a thickness of at least about 1 mm, or from about 1 to about 10 mm or from about 1 to about 5 mm;
1.5. Any of the preceding compositions in the form of a tablet or bead having a surface area of about 0.55 to about 9.5 square centimeters (cm²), or about 0.9 cm2 to about 5 cm², or from about 0.9 cm² to about 3 cm²;
1.6. Any of the preceding compositions wherein the inorganic salt of peroxymonosulfate is an alkaline earth metal salt or a mixture thereof;
1.7. Any of the preceding compositions wherein the inorganic salt of peroxymonosulfate is sodium peroxymonosulfate, potassium peroxymonosulfate, or ammonium peroxymonosulfate
1.8. Any of the preceding compositions wherein the inorganic salt of peroxymonosulfate is potassium peroxymonosulfate in an amount of about 0.5% to about 50%, or about 0.75% to about 40%, or about 0.75% to about 37%, or about 7.5% to about 15%, by weight of the total composition;
1.9. Any of the preceding compositions, e.g. a tablet, wherein the disintegrating agent is selected from natural starches, such as maize starch, potato starch; directly compressible starches such as starch 1500; modified starches such as carboxymethyl starches and sodium starch glycolate; starch derivatives such as amylose; cross-linked polyvinylpyrrolidones, such as crospovidones; modified celluloses such as cross-linked sodium carboxymethylcelluloses; alginic acid; sodium alginate; microcrystalline cellulose; methacrylic acid-divinylbenzene copolymer salts; light silicic anhydride; calcium silicate; magnesium metasilicate aluminate; carboxymethyl cellulose; and mixtures thereof;
1.10. Any of the preceding compositions wherein the disintegrating agent is crosslinked polyvinylpyrrolidone;
1.11. Any of the preceding compositions, e.g. a tablet, comprising a binder wherein the binder is selected from starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, polyvinylpyrrolidone, pectins, alginates, polyacrylamides, polyvinyloxoazolidone, polyvinyl alcohols and mixtures thereof;
1.12. Any of the preceding compositions wherein the binder is present in the composition in an amount of from 10% by weight to about 60% by weight, or from about 15% by weight to about 50% by weight, or from about 25% by weight to about 40% by weight.
1.13. Any of the preceding compositions wherein the buffering agent is an anhydrous carbonate such as sodium carbonate, a sesquicarbonate, a bicarbonate such as sodium bicarbonate, a silicate, a bisulfate, a citrate, a phosphate such as monopotassium phosphate and dipotassium phosphate, or a combination thereof in an amount of about 5.0% to about 35%, or about 10% to about 30%, or about 15% to about 25%, by weight of the total composition;
1.14. Any of the preceding compositions which is effervescent and contains an effervescent acid and an effervescent base;
1.15. The immediately preceding composition wherein the effervescent acid is citric acid, ascorbic acid, malic acid, adipic acid, tartaric acid, fumaric, succinic acid, sodium acid pyrophosphate, lactic acid, hexamic acid, citraconic anhydride, glucono-D-lactone, succinic anhydride, potassium bitartrate, acid citrate salts, sodium dihydrogen phosphate, disodium dihydrogen phosphate, sodium acid sulfite, and combinations thereof, and is present in the composition in an amount of from 10% by weight to about 60% by weight, from about 15% by weight to about 50% by weight, or even from about 25% by weight to about 40% by weight;
1.16. The immediately preceding two compositions wherein the effervescent base is sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide and mixtures thereof, and is present in the composition in an amount of from 10% by weight to about 60% by weight, or from about 15% by weight to about 50% by weight, or from about 25% by weight to about 40% by weight;
1.17. Any of the preceding compositions comprising a lubricant in an amount from about 1% by weight to about 15% by weight, or from about 1% by weight to about 12% by weight, or from about 2% by weight to about 10% by weight, or from about 3% by weight to about 8% by weight;
1.18. Any of the preceding compositions comprising a disintegrating agent in an amount of about 0.5% to about 20%, or about 0.75% to about 10%, or about 1% to about 5%, or about 1% to about 3%, by weight of the total composition;
1.19. Any of the preceding compositions containing an additional whitening agent;
1.20. Any of the preceding compositions additionally comprising one or more flavor agents, one or more fillers, one or more surfactants, one or more color agents, or any combination of two or more thereof
1.21. Any of the preceding compositions, wherein the composition is powder which consists essentially of the inorganic salt of peroxymonosulfate and one or more buffering agents.
1.22. Composition 1.21, wherein the composition is a powder and the one or more buffering agents are sodium carbonate, sodium bicarbonate, or a mixture thereof.

The following non-limiting examples are to illustrate the invention but should not be interpreted as a limitation thereon.

### EXAMPLES

### Example 1

### Effervescent Tablet

TABLE 1
Formula A

| Ingredient | Weight % |
|---|---|
| Potassium peroxymonosulfate | 15 |
| Sodium Bicarbonate | 25 |
| Sodium Carbonate | 10 |
| Citric Acid | 25 |
| Crosslinked Polyvinylpyrrolidone | 3 |
| Flavor | 1.9 |
| Polyethylene Glycol | 3 |
| Sodium Lauryl Sulfate | 3 |
| Color | 0.1 |
| Sodium benzoate | 2 |
| Sucralose | 2 |
| Sorbitol | 10 |

### Example 2

### Non-Effervescent Tablet

TABLE 2
Formula B

| Ingredient | Weight % |
|---|---|
| Potassium peroxymonosulfate | 15 |
| Sodium Bicarbonate | 25 |
| Sodium Carbonate | 10 |
| Sodium sulfate | 8 |
| Crosslinked Polyvinylpyrrolidone | 3 |
| Flavor | 1.9 |
| Polyethylene Glycol | 3 |
| Sodium Lauryl Sulfate | 3 |
| Color | 0.1 |
| Sodium benzoate | 2 |
| Sucralose | 2 |
| Sorbitol | 10 |

Tablets are prepared using the ingredients of Formula A or B by compression molding.

### Example 3

Tablets having the composition of Formula A and Formula B, respectively, are added to a mouthrinse base at 23° C. The mouthrinse base has the following formula:

TABLE 3

| Ingredient | Weight % |
|---|---|
| Surfactant | 0.1-2% |
| Sweetener | 0.0005-0.02% |
| Fluoride | 0.01-0.05% |
| Preservative | 0.05-2% |
| Humectant | 5-20% |
| Flavor | 0.05-0.6 |
| Color | 0.001-0.05% |
| Water | Balance |

### Example 4

Whitening efficacy was tested in a mouthwash type setup. MPS was added to an aqueous solution (50 mM phosphate buffer, pH 6.8) and tested versus placebo (buffer alone) and commercial 2% hydrogen peroxide mouthrinse (pH 5.2). The results are shown in Table 4.

TABLE 4

| | Placebo | 1.0% MPS | 2% HP Rinse |
|---|---|---|---|
| Δ W | −0.308 ± 0.50 | −2.849 ± 0.22 | −3.554 ± 0.87 |

Results indicate that the MPS results are on par with the 2% hydrogen peroxide rinse even with the short 1 min exposure times. The experiment was conducted at room temperature with shaking. Active oxygen of MPS at each exposure time=0.047% and theoretical active oxygen of 2% HP=0.94%.

### Example 5

Four concentrations of MPS in pH 7.5-8 phosphate buffer are compared to 0.1% hydrogen peroxide in pH 7.5-8 phosphate buffer in an in vitro testing procedure using coffee-stained bovine teeth.

The bovine teeth have initial L values of 57-65. The teeth are submerged in deionized water, then removed and soaked for 2 minutes in 20 mL of the test solution. The teeth are then rinsed three times with deionized water. Each such treatment cycle is repeated fourteen times, and after every two cycles values for L*, a* and b* are measured. These figures are used to calculate a whiteness score, W*, which is a measure of overall color change relative to pure white:

W*=((a*)²+(b*)²+(L*−100)²)^{1/2}

ΔW*=W*(treated)−W*(baseline)

The results are shown in the Table 5 below.

TABLE 5

| Treatment | 0 | 2 | 4 | 6 | 8 | 10 | 12 | 14 |
|---|---|---|---|---|---|---|---|---|
| 0.5% MPS | 0.00 | 4.13 | 6.06 | 7.90 | 8.49 | 8.74 | 9.08 | 9.04 |
| 1% MPS | 0.00 | 3.76 | 4.41 | 5.78 | 6.53 | 7.04 | 8.15 | 8.27 |
| 2% MPS | 0.00 | 4.59 | 6.26 | 8.07 | 8.67 | 8.85 | 9.14 | 9.26 |
| 0.1% HP | 0 | 0.15 | 0.22 | 0.38 | 0.6 | 0.69 | 0.73 | 1.01 |

0.5% MPS contains substantially the same active oxygen content as 0.1% hydrogen peroxide. Yet, the results demonstrate that all three concentrations of MPS significantly outperform the whitening effect achieved using hydrogen peroxide. Moreover, higher concentrations of MPS are permitted in oral care compositions due to the lower risk of soft tissue irritation, whereas higher concentration of hydrogen peroxide can cause significant irritation of the oral cavity.

### Example 6

A powdered MPS composition is prepared for use as an additive to a non-whitening or therapeutic mouthwash. The MPS composition contains 47 wt % sodium bicarbonate, 30 wt % sodium carbonate and 23 wt % Caroat whitening agent (containing 47 wt % MPS). This powdered composition can be supplied in a sachet or compressed into a tablet for addition to a mouthwash.

1 gram of the MPS powder formulation is dissolved in 10 mL of the non-whitening mouthwash formulation shown in Table 6 below, resulting in a 1% solution of MPS. The resulting instant whitening mouthwash is compared to a comparative 2% hydrogen peroxide whitening mouthwash composition using the procedure described in Example 5. The comparative composition is shown in Table 7 below. In addition, 1 gram of the MPS powder formulation is also added to 10 mL of the 2% hydrogen peroxide mouthwash for demonstration of an additive effect.

TABLE 6

| Ingredient | Weight % |
|---|---|
| Water | q.s. |
| Polymers | 0.4 |
| CETYLPYRIDINIUM | 0.075 |
| CHLORIDE | |
| Preservative | 0.05 |
| Humectants | 20 |
| Flavor and Color | 0.186 |
| Buffers | 0.022 |

TABLE 7

| Ingredient | Weight % |
|---|---|
| Water | q.s. |
| Buffers | 0.45 |
| Humectants | 20 |
| Surfactants | 1.0 |
| Hydrogen Peroxide (35%) | 5.71 |
| Polymers | 2.97 |
| Flavor/Sweetener | 0.25 |

The comparative HP mouthwash is compared to the MPS mouthwash and the MPS/HP mouthwash in side-by-side experiments. The results are shown in Tables 8 and 9 below.

TABLE 8

| Treatment | 0 | 2 | 4 | 6 | 8 | 10 | 12 | 14 |
|---|---|---|---|---|---|---|---|---|
| 1% MPS MW | 0.00 | 2.2 | 3.1 | 4.0 | 4.7 | 5.5 | 6.2 | 6.8 |
| 2% HP MW | 0.00 | 1.1 | 1.5 | 1.9 | 1.9 | 2.1 | 2.3 | 2.3 |

TABLE 9

| Treatment | 0 | 2 | 4 | 6 | 8 | 10 | 12 | 14 |
|---|---|---|---|---|---|---|---|---|
| 1% MPS/2% HP MW | 0.00 | 3.8 | 5.8 | 6.7 | 7.4 | 8.1 | 8.7 | 8.8 |
| 2% HP MW | 0.00 | 1.1 | 1.7 | 2.4 | 2.5 | 2.9 | 3.2 | 3.5 |

The results demonstrate that an instant 1% MPS mouthwash significantly out-performs the whitening effect achieved using a comparable 2% hydrogen peroxide mouthwash. The results further demonstrate a significant and unexpected additive whitening effect when MPS is added to a hydrogen peroxide mouthwash.

1. A solid composition comprising:
(a) a tooth whitening effective amount of an inorganic salt of peroxymonosulfate,
(b) a buffering agent, and optionally
(c) a disintegrating agent;
wherein the solid composition completely dissolves in water.

2. The composition of claim 1 wherein the composition dissolves in 1 minute or less, 50 seconds or less, 40 seconds or less, 30 seconds or less, or 20 seconds or less, when added to 15 ml of water.

3. The composition of claim 1 in the form of a tablet.

4. The composition of claim 1 wherein the composition contains no water or water in an amount of less than 4% by weight, or less than 3% by weight, or less than 2% by weight, or less than 1% by weight, or less than 0.5% by weight, or about 0.001% to about 4% by weight, or about 0.0001% to about 0.5% by weight or about 0.0001% to about 0.1% by weight.

5. The composition of claim 1 additionally comprising a binder.

6. The composition of claim 1 in the form of a tablet having weights from about 0.05 gram to about 5 gram, or from about 100 milligrams (mg) to about 1000 mg, or from about 100 mg to about 500 mg, or from about 200 mg to about 400 mg, a diameter of at least 5 millimeters (mm), or from about 5 mm to about 50 mm, or from about 5 mm to about 30 mm or from about 5 mm to about 20 mm, and a thickness of at least about 1 mm, or from about 1 to about 10 mm or from about 1 to about 5 mm.

7. The composition of claim 1 wherein the inorganic salt of peroxymonosulfate is potassium peroxymonosulfate in an amount of about 0.5% to about 50%, or about 0.75% to about 40%, or about 0.75% to about 37%, or about 7.5% to about 15%, by weight of the total composition.

8. The composition of claim 1 wherein the disintegrating agent is selected from natural starches, such as maize starch, potato starch; directly compressible starches such as starch 1500; modified starches such as carboxymethyl starches and sodium starch glycolate; starch derivatives such as amylose; cross-linked polyvinylpyrrolidones, such as crospovidones; modified celluloses such as cross-linked sodium carboxymethylcelluloses; alginic acid; sodium alginate; microcrystalline cellulose; methacrylic acid-divinylbenzene copolymer salts; light silicic anhydride; calcium silicate; magnesium metasilicate aluminate; carboxymethyl cellulose; and mixtures thereof; in an amount of about 0.5% to about 20%, or about 0.75% to about 10%, or about 1.0% to about 5%, by weight of the total composition.

9. The composition of claim 1 wherein the disintegrating agent is crosslinked polyvinylpyrrolidone.

10. The composition of claim 1 comprising a polymeric binder wherein the polymeric binder is selected from starches, natural gums, (e.g., xanthan gum), cellulose gums, microcrystalline cellulose, maltodextrins, methylcellulose, cellulose ethers, sodium carboxymethylcellulose, ethylcellulose, gelatin, polyethylene glycol, polyvinylpyrrolidone, pectins, alginates, polyacrylamides, polyvinyloxoazolidone, polyvinyl alcohols and mixtures thereof; and is present in the composition in an amount of from 10% by weight to about 60% by weight, or from about 15% by weight to about 50% by weight, or from about 25% by weight to about 40% by weight.

11. The composition of claim 1 wherein the buffering agent is an anhydrous carbonate such as sodium carbonate, a sesquicarbonate, a bicarbonate such as sodium bicarbonate, a silicate, a bisulfate, a citrate, a phosphate such as monopotassium phosphate and dipotassium phosphate, or a combination thereof in an amount of about 5% to about 35%, or about 10% to about 30%, or about 15% to about 25%, by weight of the total composition.

12. The composition of claim 1 which is effervescent and contains an effervescent acid and an effervescent base.

13. The composition of claim 10 wherein the effervescent acid is citric acid, ascorbic acid, malic (Currently Amended) acid, adipic acid, tartaric acid, fumaric, succinic acid, sodium acid pyrophosphate, lactic acid, hexamic acid, citraconic anhydride, glucono-D-lactone, succinic anhydride, potassium bitartrate, acid citrate salts, sodium dihydrogen phosphate, disodium dihydrogen phosphate, sodium acid sulfite, and combinations thereof, and is present in the composition in an amount of from 10% by weight to about 60% by weight, from about 15% by weight to about 50% by weight, or even from about 25% by weight to about 40% by weight; and the effervescent base is sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide and mixtures thereof, and is present in the composition in an amount of from 10% by weight to about 60% by weight, or from about 15% by weight to about 50% by weight, or from about 25% by weight to about 40% by weight.

14. The composition of claim 1 further comprising a lubricant in an amount from about 1% by weight to about 15% by weight, or from about 1% by weight to about 12% by weight, or from about 2% by weight to about 10% by weight, or from about 3% by weight to about 8% by weight.

15. The composition of claim 1 additionally comprising one or more additional whitening agents, one or more flavor agents, one or more fillers, one or more surfactants, one or more color agents, or any combination of two or more thereof.

16. A method for whitening teeth comprising mixing the solid composition of claim 1 into water or a mouthrinse base until the composition dissolves, followed by rinsing an oral cavity containing teeth with the mouthrinse.
